(19) United States
(12) Patent Application Publication
(10) Pub. No.: US 2024/0200152 A1
(43) Pub. Date: Jun. 20, 2024

(54) PRIMER SET, REAGENT COMPOSITION AND METHOD FOR THE DETECTION OF METHICILLIN-RESISTANT STAPHYLOCOCCUS AUREUS (MRSA)

(71) Applicant: GENOMTEC S.A., Wroclaw (PL)

(72) Inventors: Miron TOKARSKI, Brzeg (PL); Malgorzata MALODOBRA-MAZUR, Wroclaw (PL)

(21) Appl. No.: 18/556,185

(22) PCT Filed: Apr. 21, 2022
(86) PCT No.: PCT/PL2022/050025

§ 371 Date: Oct. 19, 2023

(30) Foreign Application Priority Data
Apr. 22, 2021 (PL) P.437660

(51) Int. Cl. C12Q 1/689; C12Q 1/6844

(52) U.S. Cl. CPC C12Q 1/689; C12Q 1/6844; C12Q 2600/156

(57) ABSTRACT

The invention relates to a set of primers, a composition of reagents and a method for detecting methicillin-resistant Staphylococcus aureus (MRSA) bacteria.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Nov. 16, 2023, is named 18556185 ST25.txt and is 4,917 bytes in size.

The invention relates to a set of primers for detecting methicillin-resistant Staphylococcus aureus (MRSA) bacteria, a method for detecting MRSA using the set of primers, and the use of the set of primers for detecting methicillin-resistant Staphylococcus aureus bacteria. The invention is applicable in medical diagnostics.

Staphylococcus aureus is a gram-positive, coagulase-positive bacterium, belonging to the Staphylococcaceae family. Staphylococcus aureus belongs to the commensal bacteria that colonize the skin, skin glands and mucous membranes, without causing disease symptoms in the host. Studies indicate that about 20% of the population are carriers of S. aureus in the nasopharynx. Staphylococcus aureus is one of the most common disease-causing bacteria in humans. Moreover, relatively more often than other pathogens, it acquires resistance to a number of antibiotics commonly used in therapy. For example, the first resistant Staphylococcus aureus strains were identified only two years after penicillin treatment was introduced. In turn, the first strain of Staphylococcus aureus resistant to the synthetic antibiotic methicillin (methicillin-resistant Staphylococcus aureus—MRSA) was identified in 1960, just one year after the introduction of methicillin in medical treatment (1959).

The molecular basis for developing resistance to a wide variety of antibiotics by Staphylococcus aureus bacteria is genetic exchange and the ability of bacteria to transfer moving parts of the genome between strains and even species.

In the case of the MRSA strain, resistance to methicillin is conditioned by the production of an alternative protein, called PBP (penicilin-binding proteins), which has an affinity to β-lactam antibiotics. The protein is encoded by the mecA gene, located on the mobile genetic element (MGE) called SCCmec (Staphylococcal Cassette Chromosome mec). The acquisition of methicillin resistance is associated with an insertion of the SCCmec cassette into the chromosome of a methicillin-sensitive bacterium.

Infections caused by the MRSA strain are characterized by a higher death rate, as well as a longer hospitalization time, and thus a higher cost of treatment. Therefore, the diagnosis of the MRSA strain, mainly to limit its spread, is an extremely important medical concern.

Laboratory diagnostics of methicillin-resistant Staphylococcus aureus is based primarily on detecting bacteria in biological material, most often in the form of a swab collected from body parts that are possible to be infected. Possible methods of detecting MRSA bacteria are bacterial culture in an appropriate medium, along with identification of the Staphylococcus aureus strain and determination of resistance/sensitivity to available antibiotics—an antibiogram. The culture tests, despite their high sensitivity and specificity, are labour-intensive and time-consuming tests. Moreover, the requirement to perform an antibiogram additionally extends the time of MRSA diagnostics.

The methods characterized by the greatest specificity and sensitivity are those involving the detection of MRSA nucleic acid in biological material (the so-called NAAT methods—Nucleic Acid Amplification Tests). The most commonly used tests in NAAT technology are Real-Time PCR-based assays. Many different tests using the Real-Time PCR technique are available on the market, but despite the fierce competition, these methods are still relatively expensive. Moreover, they require highly specialized personnel, expensive devices, and the isolation of genetic material from the patient's sample is necessary. Moreover, since cyclic heating and cooling of the reagents is necessary, this method is long, and the devices used consume relatively large amounts of energy to carry out this process.

Isothermal methods, including the LAMP (Loop-mediated isothermal amplification) method, are methods that allow to accelerate the diagnostic process and reduce the cost of energy needed to perform the analysis. Moreover, according to the literature data, these methods are characterized by higher sensitivity and specificity than the aforementioned Real-Time PCR technique, they are also much faster. Their isothermal course does not require specialized equipment.

Due to the low equipment requirements, isothermal methods are an ideal diagnostic solution for primary care units (POCT—point-of-care testing), where the test can be performed in the practice of a general practitioner or specialist doctor (gynecologist, urologist) at the first contact of a patient with the doctor. This solution allows for a quick diagnostic test (in no more than 15 minutes), which allows for selection of a targeted therapy during the very first visit. This is especially important in the case of the systemic infection (so-called sepsis) with the MRSA bacterium, which can lead to death in a very short time, and where prompt diagnosis and early treatment initiation are extremely important. On the other hand, the use of freeze-dried reagents allows the tests to be stored at room temperature, without the need to freeze the diagnostic tests.

The use of primers in the LAMP method for the diagnosis of MRSA is known from the patent applications published so far: WO2019132444A1; U.S. Ser. No. 10/370,705B2; US20180094292A1; JP2018068315A; US20170114393A1; EP2099936B1; EP2850205B1; CN105671146A; CN111094595A; WO2014073858A1; CN111868258A; JP2006271370A; KR102126429B1. The LAMP method is disclosed, for example, in patent specifications WO0028082, WO0224902. The above-mentioned patent applications in most cases do not describe the sensitivity and detection limit of MRSA bacteria. The detection method in some of the above-mentioned patent applications does not allow for quantitative measurement, and the detection is of the end-point type, using agarose gel electrophoresis or other markers based on the colour change of the reaction mixture upon a positive result of the amplification reaction. In some of the patents mentioned, an indirect measurement based on the concentration of magnesium ions was used. Some patent applications are implemented in the Real-Time technology, which enables quantitative measurement, but the detection method is based on molecular probes labelled with fluorescent dyes, which significantly increases the costs of the analysis. Other technological solutions of the detection are based on the so-called blocked primers. Moreover, in the described patent applications, the analysis time and waiting for a positive result is about 60 minutes. Besides, most of the kits developed and described above are not applicable in POCT diagnostics, and their main application is in laboratories.

Therefore, there is still a need to provide a diagnostic method using appropriately refined sets of primers used for the diagnosis of MRSA with the LAMP method, intended for use in point-of-care testing, which allows the detection of bacteria with a very low detection limit (≥10 copies/reaction) in a short time (≤20 min). Unexpectedly, the above problem was solved by the present invention.

The first subject of the invention is a set of primers for amplifying the nucleotide sequence of the mecA gene of MRSA bacteria, characterized in that it contains a set of internal primers with the following nucleotide sequences a) and b), as well as a set of external primers containing the following nucleotide sequences c) and d) specific for a selected fragment the mecA gene of MRSA bacteria:
a) 5′ GAAGGTGTGCTTACAAGTGCTAATA 3′—(nucleic sequence SEQ ID NO: 3 or its reverse and complementary sequence), linked from the 3′ preferably by TTTT bridge, to the sequence end, 5′ CAACATGAAAAATGATTATGGCTC 3′—(nucleic sequence SEQ ID NO: 4 or its reverse and complementary sequence)
b) 5′ TGACGTCTATCCATTTATGTATGGC 3′—(nucleic sequence SEQ ID NO: 5 or its reverse and complementary sequence), linked at the 3′ end, preferably by TTTT bridge, to the sequence 5′ AGGTTCTTTTTTTATCTTCGGTTA 3′—(nucleic sequence SEQ ID NO: 6 or its reverse and complementary sequence)
c) 5′ TGATGCTAAAGTTCAAAAGAGT 3′ nucleic sequence SEQ ID NO: 1 or its reverse and complementary sequence, and
d) 5′ GTAATCTGGAACTTGTTGAGC 3′ nucleic sequence SEQ ID NO: 2 or its reverse and complementary sequence.
   In a preferred embodiment of the invention, the primer set comprises a loop primer sequence containing a nucleic sequence complementary to the mecA gene of MRSA bacteria SEQ ID NO: 7—5′ CCTGTTTGAGGGTGGATAGCAGTAC 3′ or sequences reverse and complementary thereof.

The second subject of the invention is a method for detecting MRSA bacteria, characterized in that a selected region of the nucleic sequence of the MRSA genome (mecA gene fragment) is amplified using a primer set as defined in the first subject of the invention, the amplification method being the LAMP method. In a preferred embodiment, the amplification is carried out with a temperature profile: 62° C., 40 min. In a further preferred embodiment of the invention, the end-point reaction is carried out with a temperature profile of 80° C., 5 min.

The third subject of the invention is a method for detecting an infection caused by the MRSA bacterium, characterized in that it comprises the detection method defined in the second subject of the invention.

The fourth subject of the invention is a kit for detecting an infection caused by the MRSA bacterium, characterized in that it comprises a set of primers as defined in the first subject of the invention.

In a preferred embodiment of the invention, the infection detection kit comprises 5.0 μl of WarmStart LAMP Master Mix. In a further preferred embodiment of the invention, individual amplification primers as defined in the first subject of the invention, the primers having the following concentrations: 0.13 UM F3, 0.13 μM B3, 1.06 μM FIP, 1.06 μM BIP, 0.26 UM LoopF; D-(+)-Trehalose dihydrate—6%; mannitol—1.25%; fluorescent marker interacting with double-stranded DNA—EvaGreen ≤1× (Biotium) or Fluorescent Dye (New England Biolabs) in the amount of ≤1 μl or Syto-13 ≤16 μM (ThermoFisher Scientific) or SYTO-82 ≤16 μM (ThermoFisher Scientific) or another fluorescent dye interacting with double-stranded DNA at a concentration that does not inhibit the amplification reaction.

The advantage of the primer sets of the invention for detecting MRSA, as well as the method for detecting MRSA infection and the method of detecting the amplification products is the possibility of using them in medical diagnostics at the point of care (POCT) in the target application with a portable genetic analyser. Freeze-drying of the reaction mixtures of the invention allows the diagnostic kits to be stored at room temperature without reducing the diagnostic parameters of the tests. In turn, the use of a fluorescent dye to detect the amplification product increases the sensitivity of the method, allows to lower the detection limit (down to 10 genome copies/reaction), as well as it enables the quantitative measurement of MRSA bacteria in the test sample.

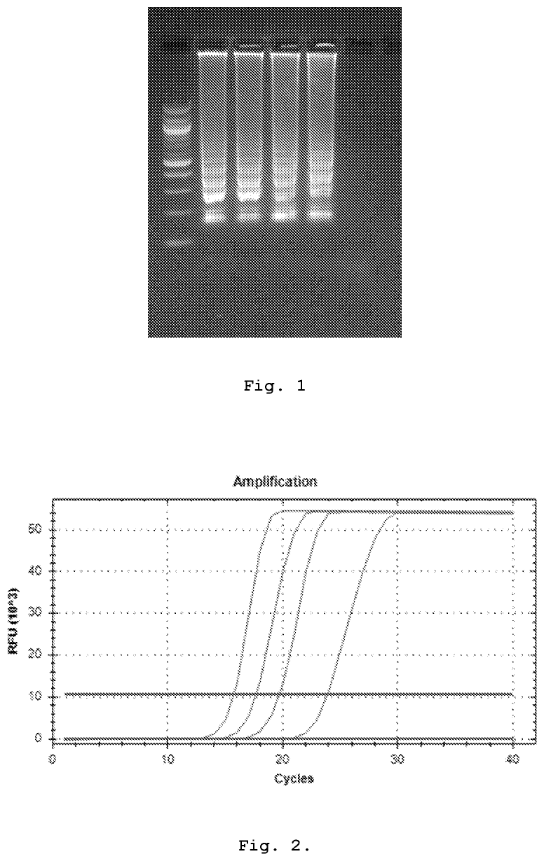

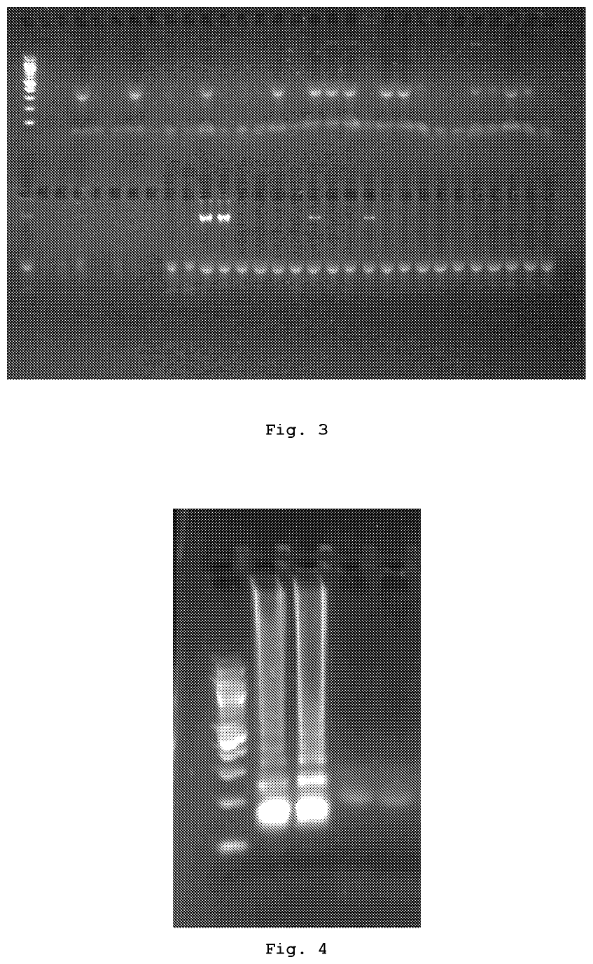

Exemplary embodiments of the invention are presented in the drawing, in which FIG. 1 shows the sensitivity characteristics of the method, where a specific signal was obtained with the template: Staphylococcus aureus Quantitative DNA (ATCC® 700699DQ™) over the range of 100-10 copies/μl, but there was no product in NTC (FIG. 1: lane 1: mass marker (Quick-Load® Purple 100 bp DNA Ladder, New England Biolabs); lane 2: 100 copies of MRSA; lane 3: 50 copies of MRSA; lane 4: 20 copies of MRSA; lane 5: 10 copies of MRSA; lane 6: NTC); FIG. 2 shows the sensitivity of the method of the invention measured by assaying a serial dilution of the Staphylococcus aureus Quantitative DNA (ATCC® 700699DQ™) standard over a range of 100-10 copies/reaction of the DNA standard, where the amplification product was measured in real time. The results of the real-time MRSA detection are shown in Table 1, giving the minimum time required to detect the fluorescence signal, while FIGS. 3 and 4 show the specificity of the method of the invention with standard matrices of a number of pathogens potentially present in the tested biological material as natural physiological flora, those which may result from co-infections or those which share similar genomic sequences (FIG. 3: lane 1: mass marker (Quick-Load® Purple 100 bp DNA Ladder, New England Biolabs); lanes 2 and 3: methicillin-sensitive Staphylococcus aureus (MSSA); lanes 4 and 5: Borrelia burgdorferi; lanes 6 and 7: Neisseria meningitidis; lanes 8 and 9: Klebsiella pneumoniae; lanes 10 and 11: Bordetella pertussis; lanes 12 and 13: Streptococcus pyogenes; lanes 14 and 15: Enterococcus faecalis; lanes 16 and 17: Enterococcus faecium; lanes 18 and 19: Pseudomonas aeruginosa; lanes 20 and 21: Moraxella catarrhalis; lanes 22 and 23: Streptococcus pneumoniae; lanes 24 and 25: Streptococcus agalactiae; lanes 26 and 27: Listeria monocytogenes; lanes 28 and 29: Legionella pneumophila; lanes 30 and 31: Haemophilus ducreyi; lanes 32 and 33: HHV5; lanes 34 and 35: Homo sapiens; lanes 36 and 37: Candida albicans; lanes 33 and 39: Escherichia coli; lanes 40 and 41: Influenza B virus; lanes 42 and 43: Influenza A H1N1 virus; lanes 44 and 45: Influenza A H3N1 virus; lanes 46 and 47: Borrelia afzelii; lanes 48 and 49: Toxoplasma gondi; lanes 50 and 51: HBV; lanes 52 and 53: Treponema pallidum; lanes 54 and 55: Heamophilus influenzae; lanes 56 and 57: SARS-COV-2; lanes 58 and 59: Actinomyces baumannii; FIG. 4: lane 1: mass marker (Quick-Load® Purple 100 bp DNA Ladder, NewEngland Biolabs); lanes 2 and 3: methicillin-resistant Staphylococcus aureus (MRSA); lanes 4 and 5: NTC).

### EXAMPLE 1. PRIMER SEQUENCES

The sequences of specific oligonucleotides used for the detection of methicillin-resistant Staphylococcus aureus genetic material using LAMP technology are presented and characterized below.
1. The MRSA mecAF3 oligonucleotide sequence: 5′ TGATGCTAAAGTTCAAAAGAGT 3′ is a sequence identical to the MRSA mecA gene (5′-3′ strand) which is 3′ end adjacent to the F2 primer.
2. The MRSA mecAB3 oligonucleotide sequence: 5′ GTAATCTGGAACTTGTTGAGC 3′ is a complementary fragment of the MRSA mecA gene (5′-3′ strand) 167 nucleotides away from the 3′ end of the oligonucleotide 1.
3. MRSA mecAF2 oligonucleotide sequence: 5′ CAACATGAAAAATGATTATGGCTC 3′ is a sequence identical to the MRSA mecA gene (5′-3′ strand) 8 nucleotides away from the 3′ end of the oligonucleotide 1.
4. The MRSA mecAB2 oligonucleotide sequence: 5′ AGGTTCTTTTTTATCTTCGGTTA 3′ is a complementary fragment of the MRSA mecA gene (5′-3′ strand) 142 nucleotides away from the 3′ end of the oligonucleotide 1.
5. The MRSA mecAF1c oligonucleotide sequence: 5′ GAAGGTGTGCTTACAAGTGCTAATA 3′ is a complementary fragment of the MRSA mecA gene (5′-3′ strand) 64 nucleotides away from the 3′ end of the oligonucleotide 1.
6. The MRSA mecAB1c oligonucleotide sequence: 5′ TGACGTCTATCCATTTATGTATGGC 3′ is a sequence identical to the MRSA mecA gene (5′-3′ strand) 92 nucleotides away from the 3′ end of the oligonucleotide 1.
7. The MRSA mecALoopF oligonucleotide sequence: 5′ CCTGTTTGAGGGTGGATAGCAGTAC 3′.

The sequences of the F1c and F2 oligonucleotides have preferably been linked by a TTTT bridge and used as FIP. The sequences of the B1c and B2 oligonucleotides have preferably been linked by a TTTT bridge and used as BIP.

### EXAMPLE 2

The method of amplifying the mecA MRSA gene using the oligonucleotides characterized in Example 1 with LAMP technology with the following composition of the reaction mixture:
5.0 μl WarmStart LAMP 2× Master Mix
0.13 UM F3
0.13 UM B3
1.06 UM FIP
1.06 UM BIP
0.26 UM LoopF
D-(+)-Trehalose dihydrate—6%
Mannitol—1.25%

Fluorescent marker interacting with double-stranded DNA—EvaGreen≤1× or Fluorescent dye 50× (New England Biolabs) in the amount of 0.5 μl or GreenFluorescent Dye (Lucigen) in the amount of ≤1 μl or Syto-13 ≤16 μM or SYTO-82 ≤16 UM or another fluorescent dye that interacts with double-stranded DNA at a concentration that does not inhibit the amplification reaction. DNA template ≥10 copies/reaction

Total reaction volume adjusted to 10 μl with DNase and RNase free water.

### EXAMPLE 3

The method of amplifying the MRSA mecA gene using the oligonucleotides characterized in Example 1 and Example 2 with LAMP technology and the composition of the reaction mixture characterized in Example 3 with the following temperature profile:
1) 62° C., 40 min
2) preferably for end-point reactions 80° C., 5 min.

### EXAMPLE 4

The method of amplification and detection of the MRSA mecA gene using the oligonucleotides characterized in Example 1 and Example 2 with LAMP technology and the composition of the reaction mixture characterized in Example 2 with the temperature profile characterized in Example 3 and the detection method described below.

A fluorescent dye is used, capable of interacting with double-stranded DNA, added to the reaction mixture in an amount of 0.5 μl EvaGreen 20×; 0.5 μl or a concentration of ≤1×; ≤16 UM respectively for GreenFluorescent Dye (Lucigen); SYTO-13 and SYTO-82 before starting the reaction, real-time and/or end-point measurement. Excitation wavelength in the range similar to the FAM dye—490-500 nm (optimally 494 nm) for EvaGreen; Fluorescent dye 50× (New England Biolabs), GreenFluorescent Dye (Lucigen); SYTO-13 dyes and 535 nm (optimally 541 nm) for the SYTO-82 dye; emission wavelength in the range 509-530 nm (optimally 518 nm) for EvaGreen; GreenFluorescent Dye (Lucigen); SYTO-13 dyes and 556 nm (optimally 560 nm) for the SYTO-82 dye, the method of detection, change recording time starting from 11 minutes from the start of the reaction for MRSA and the negative control.

### EXAMPLE 5

The method of preparation and freeze-drying of reagents for detecting the amplification and detection of the MRSA mecA gene using the oligonucleotides characterized in Example 1 and Example 2 with the LAMP technology and the composition of the reaction mixture characterized in Example 2 with the temperature profile characterized in Example 3 and the detection method described in Example 4.

### EXAMPLE 6. DESCRIPTION OF THE FREEZE-DRYING PROCESS

The reaction components were mixed according to the composition described in Example 2, except the template DNA, to a total volume of 10 μl. The mixture was transferred to 0.2 ml tubes and subjected to the freeze-drying process according to the parameters below.

The mixture placed in test tubes was pre-cooled to −80° C. for 2 hours. Then the freeze-drying process was carried out at the temperature of −80° C. for 3 hours under the pressure of 5⁻² mBar.

### EXAMPLE 7. SENSITIVITY OF THE METHOD

The sensitivity was determined by assaying serial dilutions of the Staphylococcus aureus Quantitative DNA (ATCCR 700699DQ™) standard with a minimum amount of 10 copies of bacteria per reaction mixture, where the product amplification was measured in real time—FIG. 2 (Real-Time LAMP for serial dilutions).

The time required to detect the emitted fluorescence for individual samples is shown in Table 1.

The characterized primers allow for the detection of MRSA bacteria by detecting the mecA gene fragment at a minimum number of 10 copies/reaction mixture.

TABLE 1
Time required to detect fluorescence for each dilution of the
Staphylococcus aureus Quantitative DNA (ATCC ®
700699DQ ™) standard.

| | Time to exceed the baseline |
|---|---|
| Sample | fluorescence [min] |
| NTC | Indefinite |
| MRSA 10 copies | 23.88 |
| MRSA 20 copies | 19.68 |
| MRSA 50 copies | 17.65 |
| MRSA 100 copies | 15.72 |

The superiority of the amplification method and the oligonucleotides described in this specification over the tests based on the Real-Time LAMP technology is due to the much higher sensitivity, which is shown in FIG. 1, and the reduction of the analysis time shown in FIG. 2.

1. A set of primers for amplifying the nucleotide sequence of the methicillin-resistant Staphylococcus aureus (MRSA) mecA gene, characterized in that it comprises a set of internal primers with the following nucleotide sequences a) and b), as well as a set of external primers comprising the following nucleotide sequences c) and d):
a) 5′ GAAGGTGTGCTTACAAGTGCTAATA 3′ (SEQ ID NO: 3) or a sequence at least 90% identical to SEQ ID NO: 3, linked from the 3′ end, preferably by a TTTT bridge, to the sequence 5′ CAACATGAAAAATGATTATGGCTC 3′ (SEQ ID NO: 4) or a sequence at least 90% identical to SEQ ID NO: 4;
b) 5′ TGACGTCTATCCATTTATGTATGGC 3′ (SEQ ID NO: 5) or a sequence at least 90% identical to SEQ ID NO: 5, linked from the 3′ end, preferably by a TTTT bridge, to the sequence 5′ AGGTTCTTTTTTTATCTTCGGTTA 3′ (SEQ ID NO: 6) or a sequence at least 90% identical to SEQ ID NO: 6;
c) 5′ TGATGCTAAAGTTCAAAAGAGT 3′ (SEQ ID NO: 1) or a sequence at least 90% identical to SEQ ID NO: 1, and
d) 5′ GTAATCTGGAACTTGTTGAGC 3′ nucleic sequence (SEQ ID NO: 2) or a sequence at least 90% identical to SEQ ID NO: 2.

2. The set of primers of claim 1, characterized in that it comprises a loopF primer complementary to the MRSA mecA gene 5′ CCTGTTTGAGGGTGGATAGCAGTAC 3′ (SEQ ID NO: 7) or a sequence at least 90% identical to SEQ ID NO: 7.

3. A method of detecting MRSA bacteria, characterized in that a selected region of the nucleic sequence of the bacterial genome is amplified using the set of primers as defined in claim 1, the amplification method being the LAMP method.

4. The method of detecting bacteria of claim 3, characterized in that the amplification is carried out with a temperature profile of: 62° C., 40 min

5. The method of claim 4, characterized in that an end-point reaction is carried out with a temperature profile of 80° C., 5 min.

6. A method for detecting infection caused by the MRSA bacterium, characterized in that it comprises the detection method as defined in claim 3.

7. A kit for detecting infection caused by the MRSA bacterium, characterized in that it comprises the set of primers as defined in claim 1.

8. The kit for detecting infection of claim 7, characterized in that it comprises 5.0 μl of WarmStart LAMP Master Mix (NEB).

9. The kit for detecting infection of claim 7, wherein the primers have the following concentrations:
primer c) at 0.13 μM,
primer d) at 0.13 μM,
primer b) at 1.06 μM, and
primer a) at 1.06 μM.

10. A method of detecting MRSA bacteria, characterized in that a selected region of the nucleic sequence of the bacterial genome is amplified using the set of primers as defined in claim 2, the amplification method being the LAMP method.

11. The method of detecting bacteria of claim 10, characterized in that the amplification is carried out with a temperature profile of:
−65.5° C., 40 min

12. The method of claim 11, characterized in that the end-point reaction is carried out with a temperature profile of 80° C., for additional 5 min.

13. A method for the detection of a MRSA bacterium infection, characterized in that it comprises the detection method of claim 10.

14. A kit for the detection of a MRSA bacterium infection, characterized in that it comprises a set of primers as defined in claim 2.

15. The infection detection kit of claim 14, characterized in that it comprises 5.0 μl of WarmStart LAMP 2× Master Mix (NEB).

16. The infection detection kit of claim 14, wherein the primers have the following concentrations:
primer c) at 0.13 μM,
primer d) at 0.13 μM,
primer b) at 1.06 μM,
primer a) at 1.06 μM, and
loop primer at 0.26 μM.

17. The infection detection kit of claim 9, comprising D-(+)-Trehalose dihydrate.

18. The infection detection kit of claim 9, comprising a fluorescent marker interacting with double-stranded DNA.

19. The infection detection kit of claim 14, comprising D-(+)-Trehalose dihydrate.

20. The infection detection kit of claim 14, comprising a fluorescent marker interacting with double-stranded DNA.


SEQUENCE LISTING (8 sequences)

SEQ ID NO: 1 - DNA, 22 bp, Artificial Sequence
  primer
tgatgctaaagttcaaaagagt

SEQ ID NO: 2 - DNA, 21 bp, Artificial Sequence
  primer
gtaatctggaacttgttgagc

SEQ ID NO: 3 - DNA, 25 bp, Artificial Sequence
  primer
gaaggtgtgcttacaagtgctaata

SEQ ID NO: 4 - DNA, 24 bp, Artificial Sequence
  primer
caacatgaaaaatgattatggctc

SEQ ID NO: 5 - DNA, 25 bp, Artificial Sequence
  primer
tgacgtctatccatttatgtatggc

SEQ ID NO: 6 - DNA, 23 bp, Artificial Sequence
  primer
aggttcttttttatcttcggtta

SEQ ID NO: 7 - DNA, 25 bp, Artificial Sequence
  primer
cctgtttgagggtggatagcagtac

SEQ ID NO: 8 - DNA, 2456 bp, Staphylococcus aureus
atgaactgattatacttaacattaaaaaagatgataacaccttctacacctccatatcacaaaaaattataacattattttgacataaatactacatttgtaatatactacaaatgtagtcttatataaggaggatattgatgaaaaagataaaaattgttccacttattttaatagttgtagttgtcgggtttggtatatatttttatgcttcaaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacacatatgagattaggcatcgttccaaagaatgtatctaaaaaagattataaagcaatcgctaaagaactaagtatttctgaagactatatcaacaacaaatggatcaaaattgggtacaagatgataccttcgttccactttaaaaccgttaaaaaaatggatgaatatttaagtgatttcgcaaaaaaatttcatcttacaactaatgaaacagaaagtcgtaactatcctctagaaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaaatgatggtatgcaacaagtcgtaaataaaacacataaagaagatatttatagatcttatgcaaacttaattggcaaatccggtactgcagaactcaaaatgaaacaaggagaaagtggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataacaaaacagtgaagcaatccgtaacgatggttgcttcactgttttattatgaattattaataagtgctgttacttctcccttaaatacaatttcttcattttcattgtatgttgaaagtgacactgtaacgagtccattttctttttttatggatttcttatttgtaatttcagcgataacgtacaatgtattacctggtatacagtttaataaatttaacgttattcatttgtgttcctgctacaacttcttctccgtatttaccttcttctacccataatttaaatgatattgaaagtgtatgcatgc